(19) United States
(12) Patent Application Publication
(10) Pub. No.: US 2026/0028401 A1
(43) Pub. Date: Jan. 29, 2026

(54) ANTI-ILT4 SINGLE-DOMAIN ANTIBODY AND USE THEREOF

(71) Applicant: SHENGHE (CHINA) BIOPHARMACEUTICAL CO., LTD., Nanjing, Jiangsu (CN)

(72) Inventors: Huabing YANG, Nanjing (CN); Liusong YIN, Nanjing (CN); Xiaoling JIANG, Nanjing (CN)

(73) Assignee: SHENGHE (CHINA) BIOPHARMACEUTICAL CO., LTD., Nanjing, Jiangsu (CN)

(21) Appl. No.: 18/997,819

(22) PCT Filed: Jul. 28, 2023
(86) PCT No.: PCT/CN2023/109704

§ 371 Date: Jan. 23, 2025

(30) Foreign Application Priority Data
Jul. 29, 2022 (WO) PCT/CN2022/108780

(51) Int. Cl. C07K 16/28

(52) U.S. Cl. CPC C07K 16/2803; C07K 2317/24; C07K 2317/52; C07K 2317/565; C07K 2317/569; C07K 2317/76; C07K 2317/92

(57) ABSTRACT

Provided is an anti-ILT4 single-domain antibody and antigen-binding fragment thereof. Also provided are a method of using it for the treatment or prevention of a disease in a subject, and a method of preparing the antibody and antigen-binding fragment thereof.

### TECHNICAL FIELD

The present invention belongs to the field of tumor immunotherapy and molecular immunology, and specifically relates to an anti-ILT4 single-domain antibody and use thereof.

BACKGROUND

ILT4 (Immunoglobulin-like transcript 4), also known as CD85D, LILRB2, LIR2,MIR10, is encoded by the LILRB2 gene and belongs to the family of activating and inhibitory immunoglobulin-like transcripts (ILTs) that regulate immune cell activation. ILT4 is a classical type I transmembrane protein with four extracellular tandem Ig-like structural domains, a 23-amino acid transmembrane structural domain, and a cytoplasmic tail with three immunoreceptor tyrosine inhibitory motifs (ITIMs). Physiologically, ILT4 is expressed mainly in innate immune cells such as monocytes, macrophages, dendritic cells (DCs) and granulocytes. Binding of the ligand to ILT4 recruits SH-2 containing SHP-1 or SHP-2 phosphatases in immune cells, thereby inhibiting calcium mobilisation and suppressing activation signals in monocytes and dendritic cells.

Studies have shown that ILT4 is also highly expressed in various solid tumors, including NSCLC, breast cancer, oesophageal cancer and pancreatic cancer. In a pancreatic endothelial cell malignant transformation model, ILT4 expression was significantly induced during a multistep carcinogenesis process. Clinically, ILT4 expression levels in tumor cells of patients with non-small cell lung cancer and breast cancer were positively correlated with poor cell differentiation, increased local lymph node metastasis, advanced cancer stage and poor patient survival. It has also been shown that ILT4 directly controls the behaviour of malignant tumor cells through: 1) promotion of tumor proliferation and growth; 2) increased tumor invasion and metastasis; 3) maintenance of an immunosuppressive tumor microenvironment; 4) generation of tolerogenic dendritic cells; 5) inhibition of the development and function of T effector cells; and 6) induction of various regulatory T-cell (Treg) subpopulations. In addition to signalling pathway-mediated regulation of tumor biology, ILT4 activates PI3K/AKT/mTOR signalling and NF-κB pathways to directly and/or indirectly regulate adaptive anti-tumor immune responses.

These studies indicated that ILT4 has a potential role as a new immune checkpoint target in tumor immunotherapy. In view of the fact that the current success rate of immune checkpoint therapy still falls short of expectations, there is an urgent need for the development of drugs targeting new and alternative checkpoint molecules/signals to improve anti-tumor immunotherapy.

### SUMMARY

For the above purposes, the present invention provides an anti-ILT4 single-domain antibody or antigen-binding fragment thereof.

In some embodiments, the single-domain antibody or antigen-binding fragment thereof comprises CDR1, CDR2 and CDR3, wherein the CDR1, CDR2 and CDR3 comprise, respectively, an amino acid sequence as shown in SEQ ID NOs: 1-3, or a sequence having at least 80% identity to the amino acid sequence of SEQ ID NOs: 1-3, or an amino acid sequence having one or more (preferably 2 or 3) conserved amino acid mutations (preferably substitutions, insertions or deletions) compared to the amino acid sequence of SEQ ID NOs: 1-3.

In some embodiments, the CDR1, CDR2 and CDR3 comprise an amino acid sequence as shown in SEQ ID NOs: 1-3, respectively.

In some embodiments, the single-domain antibody or antigen-binding fragment thereof comprises an amino acid sequence as shown in SEQ ID NOs: 4, 6, or a sequence having at least 80% identity to the amino acid sequence of SEQ ID NOs: 4, 6, or an amino acid sequence having one or more (preferably 2 or 3) conserved amino acid mutations (preferably substitutions, insertions or deletions) compared to the amino acid sequence of SEQ ID NOs: 4, 6.

In some embodiments, the single-domain antibody or antigen-binding fragment thereof further comprises an immunoglobulin Fc region.

In some embodiments, the dissociation constant KD between the single-domain antibody or antigen-binding fragment thereof and ILT4 is less than 10 nM.

In some embodiments, the dissociation constant KD between the single-domain antibody or antigen-binding fragment thereof and ILT4 is less than 1 nM.

In some embodiments, the single-domain antibody or antigen-binding fragment thereof comprises chimeric, humanized or fully-humanized.

The present invention provides a polynucleotide encoding the single-domain antibody or antigen-binding fragment thereof as described in any one of the above.

In some embodiments, the polynucleotide is selected from the polynucleotide sequences corresponding to the sequences SEQ ID NOs: 5, 8.

The present invention provides a recombinant vector, transgenic cell line, phage, recombinant bacterium or viral vector, wherein the recombinant vector, transgenic cell line, phage, recombinant bacterium or viral vector comprises the polynucleotide as described in any one of the above.

The present invention provides an isolated host cell containing the recombinant vector, transgenic cell line, phage, recombinant bacterium or viral vector as described above.

In some embodiments, the host cell is a prokaryotic cell. In some embodiments, the host cell is a eukaryotic cell. In some embodiments, the eukaryotic cell is a mammalian cell. In some embodiments, the mammalian cell is a CHO cell.

The present invention provides a method of antibody expression, wherein a recombinant vector, transgenic cell line, phage, recombinant bacterium or viral vector as described above is used to express an antibody protein in the host cell as described in any of the above.

The present invention provides use of the single-domain antibody or antigen-binding fragment thereof as described above in the preparation of a drug, wherein the medicament comprises the single-domain antibody or antigen-binding fragment thereof and a chemotherapeutic treatment for the treatment of a tumor in a human patient, wherein the single-domain antibody or antigen-binding fragment thereof and the chemotherapeutic treatment are formulated in such a way as to provide a therapeutic effect that is greater than the sum of the effects of the respective effects.

The present invention provides a construct, wherein the construct comprises the single-domain antibody or antigen-binding fragment thereof as described above, and a second portion selected from a second antibody or antigen-binding fragment thereof, a detectable marker, a drug, a gold nanoparticle/nanorod, a magnetic nanoparticle, a viral capsid protein or a viral particle, a radionuclide, a liposome, a chemotherapeutic agent, or a combination thereof.

In some embodiments, the second antibody or antigen-binding portion thereof has a binding specificity that is different from that of the single-domain antibody or antigen-binding fragment thereof as described in any one of the above. In some embodiments, the antigen of the second antibody or antigen-binding portion thereof is selected from a tumor associated antigen (TAA) or an immune checkpoint.

In some embodiments, the detectable marker is a radionuclide.

In some embodiments, the drug is selected from the group consisting of: a toxin, a cytokine, or an enzyme.

The present invention provides a pharmaceutical composition comprising the single-domain antibody or antigen-binding fragment thereof as described in any one of the above.

In some embodiments, the composition further comprises an additional therapeutic agent. In some embodiments, the additional therapeutic agent is an immunotherapeutic agent.

The present invention provides use of the pharmaceutical composition as described above in the preparation of a drug for the treatment of a disease associated with ILT4, wherein the disease comprises a tumor or an autoimmune disease.

The present invention provides use of the single-domain antibody or antigen-binding fragment thereof as described above in the preparation of a drug for the treatment and/or prevention and/or diagnosis of a disease.

In some embodiments, the disease is selected from astrocytoblastoma of the human brain, human pharyngeal head cancer, adrenal gland tumor, AIDS-associated cancer, vesicular soft tissue sarcoma, astrocytoma, bladder cancer, bone cancer, brain and spinal cord cancer, metastatic brain tumor, breast cancer, carotid body tumor, cervical cancer, chondrosarcoma, chordoma, renal smoky cell carcinoma, clear cell carcinoma, colorectal cancer, colorectal carcinoma, connective tissue-promoting proliferative small round-cell neoplasm, ventricular pleocytoma, Ewing's tumor, Extraosseous mucinous chondrosarcoma, osteofibrous dysplasia, osteofibrous dysplasia, gallbladder or bile duct cancer, gastric cancer, gestational trophoblastic disease, germ cell tumor, head and neck cancer, hepatocellular carcinoma, pancreatic islet cell tumor, Kaposi's sarcoma, renal carcinoma, leukaemia, liposarcoma/malignant lipomatous tumor, hepatocellular carcinoma, lymphoma, lung carcinoma, adult neurotubular carcinoma, melanoma, meningioma, multiple endocrine neoplasia, multiple myeloma, myelodysplastic syndrome, adult neuroblastoma, neuroendocrine tumor, ovarian cancer, pancreatic cancer, papillary thyroid cancer, parathyroid adenoma, paediatric cancer, peripheral nerve sheath tumor, pheochromocytoma, pituitary tumor, prostate cancer, posterior uveal melanoma, metastatic renal carcinoma, rhabdomyosarcoma, rhabdomyosarcoma, sarcoma, skin cancer, soft-tissue sarcoma, squamous cell carcinoma, synovial sarcoma, testicular cancer, thymic carcinoma, thymoma, metastatic thyroid cancer or uterine cancer.

### BRIEF DESCRIPTION OF THE DRAWINGS

FIG. 1: Diagram of the binding curve of the antibody of the present invention to the ILT4 protein.

FIG. 2: Diagram of the effect of single-site binding of the antibody of the present invention to CHO-ILT4 cells.

FIG. 3: Diagram of the multi-point binding curve of the antibody of the present invention to CHO-ILT4 cells.

FIG. 4: Diagram of the effect of single-site binding of the antibody (chimeric antibody) of the present invention to CHO-ILT4 cells.

FIG. 5: Diagram of multi-point binding curve of the antibody (chimeric antibody) of the present invention to CHO-ILT4 cells.

FIG. 6: Diagram of the effect of the antibody (chimeric antibody) of the present invention in blocking the binding of ILT4 to HLA-G.

FIG. 7: Diagram of the M1 activation experiment with the antibody (chimeric antibody) of the present invention.

FIG. 8: Diagram of the multi-point binding curve of the antibody (humanized antibody) of the present invention to CHO-ILT4 cells.

FIG. 9: Diagram of the binding curve of ILT4 to HLA-G blocked by the antibody (humanized antibody) of the present invention.

FIG. 10: Diagram of the M1 activation experiment for the antibody (humanized antibody) of the present invention.

### DETAILED DESCRIPTION

Unless otherwise defined, all technical and scientific terms used herein have the same meaning as is commonly understood by one of ordinary skill in the art to which this application belongs. While methods and materials similar or equivalent to those described herein may be used in the practice or testing of this application, suitable methods and materials are described below. In the event of a contradiction, the patent specification prevails.

There are many methods/systems in the art to define and describe CDR. These systems and/or definitions have been developed and refined for many years, including Kabat, chothia, IMgt, ABM and contact. Kabat is the most commonly used and defines CDRs based on sequence variability; Chothia defines CDRs based on sequence variability based on the position of the structural loop region; the IMGT system defines CDRs based on sequence variability and position within the variable domain structure; AbM is based on Oxford Molecular's AbM antibody modelling software and is a compromise between Kabat and Chothia; Contact defines the CDR based on the analysis of complex crystal structures and is similar to Chothia in several respects. Numbering of amino acid positions (e.g. amino acid residues in the Fc region) and target regions (e.g. CDR) in the present invention, using the Kabat system.

The term “single-domain antibody” as used herein refers to a fragment containing a single variable domain of an antibody, also known as a nanobody, which selectively binds to a specific antigen as well as a complete antibody. Single-domain antibody is much smaller compared to the 150-160 kDa mass of a complete antibody, which is only about 11-15 kDa.

The term “chimeric antibody” as used herein refers to an immunoglobulin or antibody whose variable region is derived from a first species and whose constant region is derived from a second species. Chimeric immunoglobulins or antibodies may be constructed, for example, by genetic engineering from immunoglobulin gene segments belonging to different species.

The term “humanized antibody” as used herein refers to an antibody that comprises at least one humanized antibody chain. The term “humanized antibody chain” refers to an antibody chain having a variable region, the variable region comprising a substantial variable framework region and complementarity decision of the human antibody. The regions (CDRs) are substantially derived from a non-human antibody (e.g., at least one CDR, two CDRs, or three CDRs). In some embodiments, the humanized antibody chain further comprises a constant region.

The term “identity” as used herein is defined as the percentage of amino acid residues in a candidate sequence that are identical to amino acid residues in a control polypeptide sequence after comparing the sequences and introducing gaps where necessary to obtain the maximum percentage sequence identity. Comparisons for the purpose of determining percentage amino acid sequence identity can be performed in a variety of ways within the skill of the art, for example, using publicly available computer software, such as BLAST software or the FASTA programme package. The term “at least 80% identity” means that the percentage of amino acid residues in the candidate sequence that are identical to amino acid residues in the control polypeptide sequence is 80% or more, including 80%, 81%, 82%, 83%, 84%, 85%, 86%, 87%, 88%, 89%, 90%, 91%, 92%, 93%, 94%, 95%, 96%, 97%, 98%, 99%, 100%.

The term “vector” as used herein, generally refers to a nucleic acid molecule capable of self-replication in a suitable host, which transfers the inserted nucleic acid molecule into and/or between host cells. The term may include vectors primarily for insertion of DNA or RNA into a cell, vectors primarily for replication of DNA or RNA, and expression vectors for transcription and/or translation of DNA or RNA. It also comprises vectors that perform more than one of these functions. An “expression vector” is a polynucleotide that can be transcribed and translated into a polypeptide when introduced into a suitable host cell.

The term “macrophage M1” is used interchangeably with “M1 macrophage” and “classically activated macrophage”. Macrophages are divided into two subpopulations, M1 macrophages and M2 macrophages, based on their function and level of inflammatory factor secretion. M1 macrophages (classically activated macrophages) are mainly activated by LPS and IFNγ, and secrete high levels of IL2 and low levels of IL10, which promote inflammation, bacteriostasis and phagocytosis. While M2 macrophages (alternatively activated macrophages) are mainly activated by IL4 inflammatory factor and inhibit M1 macrophages by secreting anti-inflammatory cytokines such as IL10, which plays a role in wound healing and tissue repair.

The term “TNFα” is used herein refers to a pro-inflammatory cytokine produced mainly by macrophages, monocytes, certain T-lymphocytes and NK cells, as well as brain cells and hepatocytes, and is involved in normal inflammatory and immune responses. The present invention examines whether macrophages are M1-type macrophages by whether they secrete TNFα.

The term “KD” as used herein refers to the dissociation equilibrium constant for a particular antibody-antigen interaction. Typically, an antibody binds antigen with a dissociation equilibrium constant (KD) of less than about 1E-8 M, e.g., less than about 1E-9 M, 1E-10 M, or 1E-11 M, or less, e.g., as determined using biofilm layer interferometry. The smaller the KD value, the greater the affinity.

The term “pharmaceutical composition” as used herein contains single-domain antibody or antigen-binding fragment thereof of the present invention. Typically, single-domain antibody or antigen-binding fragments thereof of the invention can be formulated in a non-toxic, inert and pharmaceutically acceptable aqueous medium. The pharmaceutical composition may be administered by conventional routes including, but not limited to, intratumoural, intraperitoneal, intravenously or topically. The pharmaceutical composition can be used to bind the ILT4 protein molecule directly and thus can be used to treat tumors. In addition, other therapeutic agents may be used concurrently.

The present invention is further described below in connection with the accompanying drawings and specific embodiments, and the protection of the present invention is not limited to the following embodiments. It should also be understood that the terms used in the embodiments of the present invention are intended to describe particular specific embodiments and are not intended to limit the scope of protection of the present invention. Without departing from the spirit and scope of the inventive conception, variations and advantages that can be thought of by those skilled in the art are included in the present invention and the scope of protection of the invention is given by the appended claims and any equivalents thereof.

### Example 1

### Immunization Library Construction

One alpaca was immunized with Human LILRB2 (Manufacturer: KACTUS, Cat: LIL-HM4B2) four times, once every two weeks, with 0.5 mg of protein per injection, plus Fuchs' complete adjuvant. Two weeks after the end of immunization, 100 mL of blood was collected for library construction and 1 mL of blood was used for immune titer detection. PBMC were isolated, and RNA was extracted and reverse transcribed into cDNA to amplify VHH for library construction.

### Example 2

### Antibody Screening

The constructed alpaca immune library were screened, and the specific VHH antibody against Human LILRB2 (manufacturer: KACTUS, Cat: LL-HM4B2) protein was enriched by pancreatic enzyme elution. The enrichment of different output sets was detected by ELISA screening. After ELISA preliminary screening, all pools were well enriched at phage level, and a total of 51 molecules with unique sequences were obtained, among which the antibody with better activity (sequence was SEQ ID NO: 4) was screened to be used as the antibody of the present invention.

### Example 3

### Binding of the Antibody of the Present Invention to ILT4 Protein

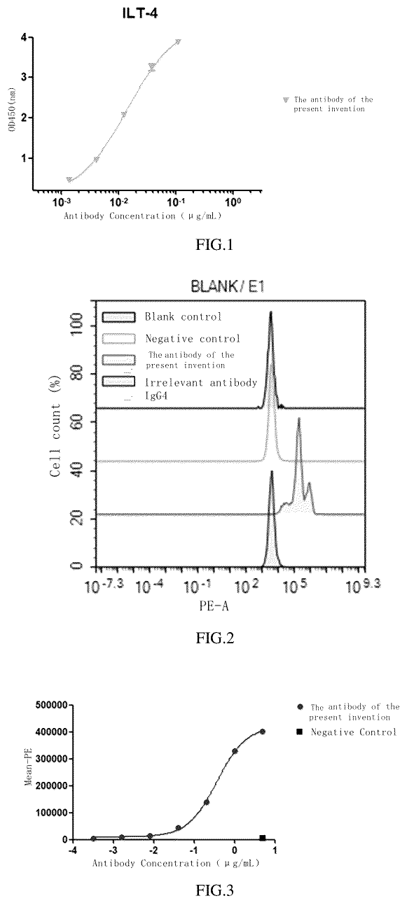

Human LILRB2 (Manufacturer: KACTUS, Cat: LIL-HM4B2) was diluted to 1 μg/mL and packed into 96-well enzyme labeled plates at 100 μL/well at 4° C. overnight. The coated solution was poured off, the plate was washed with 1×PBST at 300 μL per well, and washed with a plate washer 3 times, and patted dry on a dust-free paper. 3% skimmed milk powder was prepared, and incubated at 37° C. at 300 μL/well for 1 h. The blocking solution was poured off, the plate was washed with 1×PBST at 300 μL per well, and washed with a plate washer 3 times, and patted dry on a dust-free paper to block the proteins. The antibody of the present invention was diluted to 1 μg/mL with 3% skimmed milk powder, and this was used as the initial concentration for 3-fold dilution, with a total of 7 gradients of dilution, and another blank well was set up, and only the dilution solution was added, and then incubated at 37° C. at 100 μL/well for 1 h. Liquid in the wells was discarded, and the plate was washed by 1×PBST at 300 μL per well, and washed by a plate washer 3 times, and patted dry on a dust-free paper. The anti-VHH-HRP secondary antibody MonoRab™ Rabbit Anti-Camelid VHH Cocktail [HRP] (Manufacturer: Genscript, Cat: A06016-200) was diluted at 1:10,000 with 3% skimmed milk powder, and incubated at 37° C. at 100 μL/well for 45 minutes. The secondary antibody liquid was poured off, and the plate was washed with 1×PBST for 300 μL per well with a plate washer six times, and patted dry on a dust-free paper. A single component TMB colour development mix (manufacturer: Solepol, CAT: PR1200) was added at 100 μL/well and reacted at 37° C. without light for 7 min. Termination solution 1M HCl (83 mL 37% concentrated hydrochloric acid +917 mL pure water) was added at 100 μL/well to terminate the colour development reaction. The binding curve of the antibody of the present invention and ILT4 protein was obtained by reading at 450 nm on the enzyme-labeled instrument, as shown in FIG. 1. It can be seen from the results that the antibody of the present invention has good binding ability with ILT4 protein.

### Example 4

### Binding of the Antibody of the Present Invention to CHO-ILT4 Cells at a Single Site

The antibody of the present invention was diluted to 5 μg/mL (final concentration 2.5 μg/mL) with diluent (PBS+2% FBS). The CHO-ILT4 cells (Manufacturer: Kang Yuan Bochuang, Cat: KC-1473) were washed twice, divided into groups, 1E5/well, and washed twice, and centrifuged to remove the supernatant. Protein and antibody were added at 100 μL/well with a total volume of 200 μL, and incubated at 4° C. for 1 h. After washing the cells, the secondary antibody MonoRab™ Rabbit Anti-Camelid VHH Cocktail [PE] (Manufacturer: Genscript, Cat: A02018-200) was added at 1:500, and incubated at 4° C. without light for 0.5 h. Afterwards, the PE fluorescence reading value was detected by a flow cytometry. The results showed that the antibody of the present invention can significantly bind specifically to CHO-ILT4 cells, as shown in FIG. 2.

### Example 5

Binding of the Antibody of the Present Invention to CHO-ILT4 cells at Multiple Sites

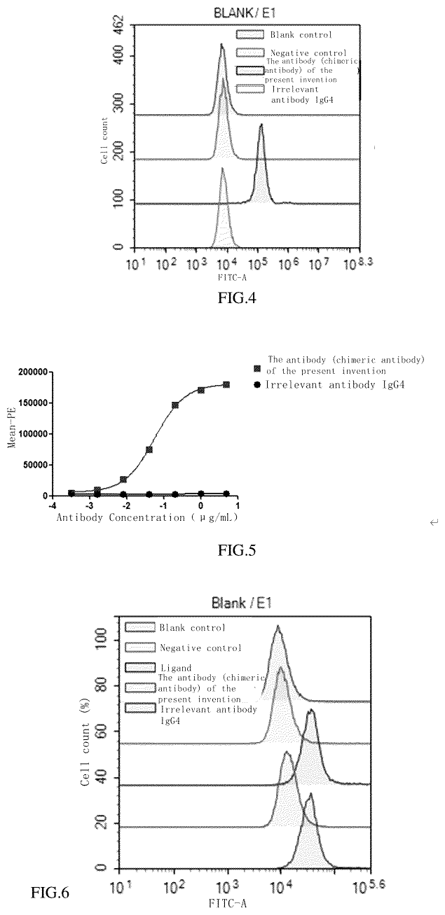

The antibody of the present invention was diluted to 5 μg/mL (final concentration 2.5 μg/mL) with diluent (PBS+2% FBS). Then this was used as the initial concentration for 5-fold dilution, with a total of 7 gradients of dilution. The CHO-ILT4 cells (Manufacturer: Kang Yuan Bochuang, Cat: KC-1473) were washed twice, divided into groups, 1E5/well, and washed twice, and centrifuged to remove the supernatant. Protein and antibody were added at 100 μL/well with a total volume of 200 μL, and incubated at 4° C. for 1 h. After washing the cells, the secondary antibody MonoRab™ Rabbit Anti-Camelid VHH Cocktail [PE] (Manufacturer: Genscript, Cat: A02018-200) was added at 1:500, and incubated at 4° C. without light for 0.5 h. Afterwards, the PE fluorescence reading value was detected by a flow cytometry. The results showed that the antibody of the present invention can significantly bind specifically to CHO-ILT4 cells with a binding EC₅₀ value of 0.36 μg/mL, as shown in FIG. 3.

### Example 6

### Binding of the Antibody (Chimeric Antibody) of the Present Invention to CHO-ILT4 Cells at a Single Site

The antibody (chimeric antibody) of the present invention (VHH and Fc sequences were SEQ ID NO: 4 and SEQ ID NO: 7, respectively) and irrelevant antibody IgG4 were diluted to 5 μg/mL (final concentration 2.5 μg/mL) with diluent (PBS+2% FBS). The CHO-ILT4 cells (Manufacturer: Kang Yuan Bochuang, Cat: KC-1473) were washed twice, divided into groups, 1E5/well, and washed twice, and centrifuged to remove the supernatant. Protein and antibody were added at 100 μL/well with a total volume of 200 μL, and incubated at 4° C. for 1 h. After washing the cells, the secondary antibody F(ab′)2 Goat anti-human IgG Fcγ Antibody was added at 1:500, and incubated at 4° C. without light for 0.5 h. Afterwards, the PE fluorescence reading value was detected by a flow cytometry. The results showed that the antibody (chimeric antibody) of the present invention can significantly bind specifically to CHO-ILT4 cells, as shown in FIG. 4.

### Example 7

### Binding of the Antibody (Chimeric Antibody) of the Present Invention to CHO-ILT4 Cells at Multiple Sites

The antibody (chimeric antibody) of the present invention and irrelevant antibody IgG4 were diluted to 5 μg/mL (final concentration 2.5 μg/mL) with diluent (PBS+2% FBS). Then this was used as the initial concentration for 5-fold dilution, with a total of 7 gradients of dilution. The CHO-ILT4 cells (Manufacturer: Kang Yuan Bochuang, Cat: KC-1473) were washed twice, divided into groups, 1E5/well, and washed twice, and centrifuged to remove the supernatant. Protein and antibody were added at 100 μL/well with a total volume of 200 μL, and incubated at 4° C. for 1 h. After washing the cells, the secondary antibody F(ab′)2 Goat anti-human IgG Fcγ Antibody was added at 1:500, and incubated at 4° C. without light for 0.5 h. Afterwards, the PE fluorescence reading value was detected by a flow cytometry. The results showed that the antibody (chimeric antibody) of the present invention can significantly bind specifically to CHO-ILT4 cells with a binding EC₅₀ value of 0.057 μg/mL, as shown in FIG. 5.

### Example 8

### Blocking the Binding of ILT4 to HLA-G by the Antibody (Chimeric Antibody) of the Present Invention

Human HLA-G biotin (Manufacturer: KATUS, Cat: HLG-HM41CTB) was diluted to 300 nM (final concentration 100 nM) with diluent (PBS+2% FBS), and the antibody (chimeric antibody) of the present invention and irrelevant antibody IgG4 were diluted to 60 μg/mL (final concentration 20 g/mL). The CHO-ILT4 cells (Manufacturer: Kang Yuan Bochuang, Cat: KC-1473) were washed twice, divided into groups, 1E5/well, and washed twice, and centrifuged to remove the supernatant. Protein and antibody were added in 50 μL/well with a total volume of 100 μL, and incubated at 4° C. for 1 h. After washing the cells, the secondary antibody SA FITC was added at 1:100, and incubated at 4° C. without light for 0.5 h. Afterwards, FITC fluorescence reading value was detected by a flow cytometry. The results showed that the antibody (chimeric antibody) of the present invention completely inhibits the binding of the ILT4 cell line to HLA-G, as shown in FIG. 6.

### Example 9

### M1 Activation Assay of the Antibody (Chimeric Antibody) of the Present Invention

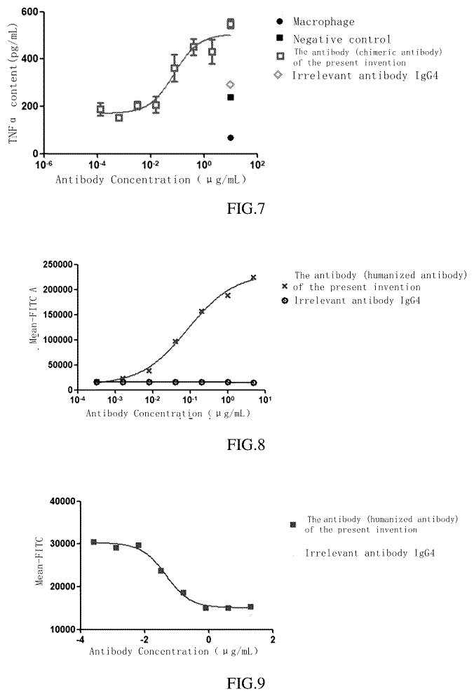

Resuscitated PBMC (ID #: PCH20201200031, CAT #: FPB004-C) were used to isolate monocytes using a monocyte isolation kit. Monocytes were resuspended with 1640 medium (with 10% inactivated FBS added) and M-CSF was added to reach a final concentration of 100 ng/mL. Cells were spread into Danish 6-well plates at a density of 2E6/mL and stimulated for 7 days. Adherent macrophages were obtained. Trypsin digestion was performed, then centrifuged, and resuspended in 1640 medium (with 10% inactivated FBS) and added to untreated Corning 96-well plates at 1E5/100 μL/well. The antibody (chimeric antibody) of the present invention and irrelevant antibody IgG4 were diluted to 20 μg/mL (final concentration 10 μg/mL) and then 5-fold diluted for a total of 8 gradients, and 50 μL/well was added to the well plate. Lipopolysaccharide was diluted to 400 ng/ml (final concentration 100 ng/ml) and 50 μL/well was added to the culture plate. The final volume was 200 μL and incubated in the incubator for 24 h. The supernatant was taken after 24 h and assayed for TNFα cytokines. The results showed that the antibody (chimeric antibody) of the present invention can effectively activate macrophage M1, as shown in FIG. 7.

### Example 10

Affinity of the Antibody of the Present Invention and the Antibody (Humanized Antibody) of the Present Invention with ILT4

AHC Biosensors (Manufacturer: ForteBio. Inc., Cat: 18-5060) were pre-wetted for 20 min using PBST buffer and added to Human LILRB2/CD85d/ILT4 Protein, His Tag (Manufacturer: KATUS, Cat: LIL-HM4B2) protein diluted to 20 μg/mL using PBST to a target protein loading thickness of 1 nm. The antibody of the present invention and the antibody (humanized antibody) of the present invention (VHH and Fc sequences were SEQ ID NO: 6 and SEQ ID NO: 7, respectively) was diluted to 100 nM using PBST and added to the above sensor, and bound for 180 s and dissociated for 200 s. The results were obtained by fitting the binding and dissociation curves of the antibody of the present invention, the antibody (humanized antibody) of the present invention and ILT4 with calculated KD values of 1.07E-10M and <1.0E-12M, respectively.

### Example 11

### Binding of the Antibody (Humanized Antibody) of the Present Invention to CHO-ILT4 Cells at Multiple Sites

The antibody (humanized antibody) of the present invention and irrelevant antibody IgG4 were diluted to 10 μg/mL (final concentration 5 μg/mL) with diluent (PBS+2% FBS). Then this was used as the initial concentration for 5-fold dilution, with a total of 7 gradients of dilution. The CHO-ILT4 cells (Manufacturer: Kang Yuan Bochuang, Cat: KC-1473) were washed twice, divided into groups, 1E5/well, and washed twice, and centrifuged to remove the supernatant. Protein and antibody were added at 100 μL/well with a total volume of 200 μL, and incubated at 4° C. for 1 h. After washing the cells, the secondary antibody FITC F(ab′)2 Goat anti-human IgG Fcγ Antibody was added at 1:500, and incubated at 4° C. without light for 0.5 h. Afterwards, TITC fluorescence reading value was detected by a flow cytometry. The results showed that the antibody (humanized antibody) of the present invention can significantly bind specifically to CHO-ILT4 cells with a binding EC₅₀ value of 0.057 μg/mL, as shown in FIG. 8.

### Example 12

### Blocking the Binding of ILT4 to HLA-G by the Antibody (Humanized Antibody) of the Present Invention

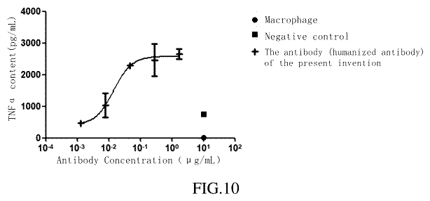

Human HLA-G biotin (Manufacturer: KATUS, Cat: HLG-HM41CTB) was diluted to 300 nM (final concentration 100 nM) with diluent (PBS+2% FBS), and the antibody (humanized antibody) of the present invention and irrelevant antibody IgG4 were diluted to 40 μg/mL (final concentration 20 μg/mL). Then this was used as the initial concentration for 5-fold dilution, with a total of 8 gradients of dilution. The CHO-ILT4 cells (Manufacturer: Kang Yuan Bochuang, Cat: KC-1473) were washed twice, divided into groups, 1E5/well, and washed twice, and centrifuged to remove the supernatant. Protein and antibody were added in 50 μL/well with a total volume of 100 μL, and incubated at 4° C. for 1 h. After washing the cells, the secondary antibody SA FITC was added at 1:500, and incubated at 4° C. without light for 0.5 h. Afterwards, FITC fluorescence reading value was detected by a flow cytometry. The results showed that the antibody (humanized antibody) of the present invention completely inhibits the binding of the ILT4 cell line to HLA-G, as shown in FIG. 9.

### Example 13

### M1 Activation Assay of the Antibody (Humanized Antibody) of the Present Invention

Resuscitated PBMC (ID #: PCH20201200031, CAT #: FPB004-C) were used to isolate monocytes using a monocyte isolation kit. Monocytes were resuspended with 1640 medium (with 10% inactivated FBS added) and M-CSF was added to reach a final concentration of 100 ng/mL. Cells were spread into Danish 6-well plates at a density of 2E6/mL and stimulated for 7 days. Adherent macrophages were obtained. Trypsin digestion was performed, then centrifuged, and resuspended in 1640 medium (with 10% inactivated FBS) and added to untreated Corning 96-well plates at 1E5/100 μL/well. The antibody (humanized antibody) of the present invention and irrelevant antibody IgG4 were diluted to 3.2 μg/mL (final concentration 1.67 μg/mL) and then 6-fold diluted for a total of 5 gradients, and 50 μL/well was added to the well plate. Lipopolysaccharide LPS was diluted to 400 ng/ml (final concentration 100 ng/ml) and 50 μL/well was added to the culture plate. The final volume was 200 μL and incubated in the incubator for 24 h. The supernatant was taken after 24 h and assayed for TNFα cytokines. The results showed that the antibody (humanized antibody) of the present invention can effectively activate macrophage M1, as shown in FIG. 10.

The protection of the present invention is not limited to the above embodiments. Without departing from the spirit and scope of the inventive concept, variations and advantages that can be thought of by those skilled in the art are included in the present invention and are protected by the appended claims.

1. An anti-ILT4 single-domain antibody or antigen-binding fragment thereof, wherein the single-domain antibody or antigen-binding fragment thereof comprises CDR1, CDR2 and CDR3, and the CDR1, CDR2 and CDR3 comprise, respectively, an amino acid sequence as shown in SEQ ID NOs: 1-3, or a sequence having at least 80% identity to the amino acid sequence of SEQ ID NOs: 1-3, or an amino acid sequence having one or more (preferably 2 or 3) conserved amino acid mutations (preferably substitutions, insertions or deletions) compared to the amino acid sequence of SEQ ID NOs: 1-3.

2. The single-domain antibody or antigen-binding fragment thereof of claim 1, wherein the CDR1, CDR2 and CDR3 comprise an amino acid sequence as shown in SEQ ID NOs: 1-3, respectively.

3. The single-domain antibody or antigen-binding fragment thereof of any one of claims 1-2, wherein the single-domain antibody or antigen-binding fragment thereof comprises an amino acid sequence as shown in SEQ ID NOs: 4, 6, or a sequence having at least 80% identity to the amino acid sequence of SEQ ID NOs: 4, 6, or an amino acid sequence having one or more (preferably 2 or 3) conserved amino acid mutations (preferably substitutions, insertions or deletions) compared to the amino acid sequence of SEQ ID NOs: 4, 6.

4. The single-domain antibody or antigen-binding fragment thereof of claims 3, wherein the single-domain antibody or antigen-binding fragment thereof further comprises an immunoglobulin Fc region.

5. The single-domain antibody or antigen-binding fragment thereof of claim 4, wherein the dissociation constant KD between it and ILT4 is less than 10 nM, preferably less than 1 nM.

6. The single-domain antibody or antigen-binding fragment thereof of of claims 5, wherein the single-domain antibody or antigen-binding fragment thereof comprises chimeric, humanized or fully-humanized.

7. (canceled)

8. (canceled)

9. (canceled)

10. (canceled)

11. (canceled)

12. (canceled)

13. (canceled)

14. (canceled)

15. (canceled)

16. (canceled)

17. A construct, wherein the construct comprises the single-domain antibody or antigen-binding fragment thereof of any one of claims 1-6, and a second portion selected from a second antibody or antigen-binding fragment thereof, a detectable marker, a drug, a gold nanoparticle/nanorod, a magnetic nanoparticle, a viral capsid protein or a viral particle, a radionuclide, a liposome, a chemotherapeutic agent, or combination thereof.

18. (canceled)

19. (canceled)

20. (Canceled)

21. (canceled)

22. Use of the single-domain antibody or antigen-binding fragment thereof of any one of claims 1-6 in the preparation of a drug for the treatment and/or prevention and/or diagnosis of a disease.

23. Use of claim 22, wherein the disease is selected from astrocytoblastoma of the human brain, human pharyngeal head cancer, adrenal gland tumor, AIDS-associated cancer, vesicular soft tissue sarcoma, astrocytoma, bladder cancer, bone cancer, brain and spinal cord cancer, metastatic brain tumor, breast cancer, carotid body tumor, cervical cancer, chondrosarcoma, chordoma, renal smoky cell carcinoma, clear cell carcinoma, colorectal cancer, colorectal carcinoma, connective tissue-promoting proliferative small round-cell neoplasm, ventricular pleocytoma, Ewing's tumor, Extraosseous mucinous chondrosarcoma, osteofibrous dysplasia, osteofibrous dysplasia, gallbladder or bile duct cancer, gastric cancer, gestational trophoblastic disease, germ cell tumor, head and neck cancer, hepatocellular carcinoma, pancreatic islet cell tumor, Kaposi's sarcoma, renal carcinoma, leukaemia, liposarcoma/malignant lipomatous tumor, hepatocellular carcinoma, lymphoma, lung carcinoma, adult neurotubular carcinoma, melanoma, meningioma, multiple endocrine neoplasia, multiple myeloma, myelodysplastic syndrome, adult neuroblastoma, neuroendocrine tumor, ovarian cancer, pancreatic cancer, papillary thyroid cancer, parathyroid adenoma, paediatric cancer, peripheral nerve sheath tumor, pheochromocytoma, pituitary tumor, prostate cancer, posterior uveal melanoma, metastatic renal carcinoma, rhabdomyosarcoma, rhabdomyosarcoma, sarcoma, skin cancer, soft-tissue sarcoma, squamous cell carcinoma, synovial sarcoma, testicular cancer, thymic carcinoma, thymoma, metastatic thyroid cancer or uterine cancer.


SEQUENCE LISTING (8 sequences)

SEQ ID NO: 1 - protein, 5 aa, synthetic construct
RYAMG

SEQ ID NO: 2 - protein, 18 aa, synthetic construct
AISWSPGDITTYADSVRG

SEQ ID NO: 3 - protein, 11 aa, synthetic construct
DPTAWGTQYDY

SEQ ID NO: 4 - protein, 121 aa, synthetic construct
EVQLVESGGGLGQAGGSLRLSCVVSGRTFSRYAMGWFRQAPGKEREFVAAISWSPGDITTYADSVRGRFTISRDNAKNTVYLQMNSLKPEDTAVYYCAADPTAWGTQYDYWGQGTQVTVSS

SEQ ID NO: 5 - DNA, 363 bp, synthetic construct
  source 1..363: mol_type = other DNA
gaggtgcagctggtggagtctgggggaggcttggggcaggctgggggctctctgagactctcctgtgtagtctctggacgcaccttcagtaggtatgccatgggctggttccgccaggctccagggaaggagcgtgagtttgtagcagctattagctggagtcctggggatatcacaacgtatgcagactccgtgaggggccgattcaccatctcccgagacaacgccaagaacacggtgtatctacaaatgaacagcctgaaacctgaggacacggccgtttattactgtgcagcagatccgacggcttggggtacgcagtatgactactggggccaggggacccaggtcaccgtctcctca

SEQ ID NO: 6 - protein, 121 aa, synthetic construct
EVQLVESGGGLVQPGGSLRLSCAVSGRTFSRYAMGWFRQAPGKEREFVAAISWSPGDITTYADSVRGRFTISRDNAKNTVYLQMNSLRAEDTAVYYCAADPTAWGTQYDYWGQGTLVTVSS

SEQ ID NO: 7 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - DNA, 363 bp, synthetic construct
  source 1..363: mol_type = other DNA
gaagtgcaactagtggaaagtggtggtggtctggtgcagcctggcggctctctgcggctgtcttgtgccgtgtctggcagaaccttttccagatatgccatgggctggttccggcaagctcctggaaaggaaagagagttcgtggccgccatctcctggagccccggcgacatcaccacctacgccgactccgtgcggggcagattcaccatctctcgcgacaacgccaagaacaccgtgtacctgcagatgaactccctgagagccgaggatacagctgtgtactactgcgctgctgatcctaccgcttggggcacccagtacgactactggggacagggcacactggtcaccgtgtccagc